(19) United States
(12) Patent Application Publication
(10) Pub. No.: US 2025/0318891 A1
(43) Pub. Date: Oct. 16, 2025

(54) OSTOMY ASSISTANCE TABLE

(71) Applicant: Melissa Gonzales, San Antonio, TX (US)

(72) Inventor: Melissa Gonzales, San Antonio, TX (US)

(21) Appl. No.: 19/175,746

(22) Filed: Apr. 10, 2025

Related U.S. Application Data
(60) Provisional application No. 63/632,877, filed on Apr. 11, 2024.

(51) Int. Cl. A61B 50/15; A47B 9/00; A47G 1/02; A61B 50/22; A61F 5/445

(52) U.S. Cl. CPC A61B 50/15; A61B 50/22; A61F 5/445; A47B 9/00; A47B 2200/005; A47G 1/02

(57) ABSTRACT

Apparatus and associated methods relate to an ostomy bag replacement system including an ostomy bag replacement table (OBRT). The OBRT may, for example, include adjustable legs operatively coupled to a bottom surface of the OBRT. The OBRT may, for example, include a top surface. The top surface may, for example, operatively couple two or more waste disposal units. A first at least one waste disposal unit may, for example, include a closed unit. A second at least one waste disposal unit may for example include an aperture. The aperture may, for example, fluidly couple tubing. The top surface may, for example, include a removable and adjustable mirror. The top surface may, for example, include an elevated surface that extends along an outer edge of the top surface. Various embodiments may advantageously provide enhanced convenience, safety, and hygiene for users managing their ostomy care routines.

### CROSS-REFERENCE TO RELATED APPLICATIONS

This application is a non-provisional application and claims the benefit of U.S. Application Ser. No. 63/632,877, titled “OSTOMY ASSISTANCE TABLE,” filed by Melissa Gonzales on Apr. 11, 2024.

This application incorporates the entire contents of the foregoing application herein by reference.

### TECHNICAL FIELD

Various embodiments relate generally to medical devices.

### BACKGROUND

Ostomy bags are medical devices used to collect waste from the body when a person has undergone surgery that diverts the normal flow of urine or stool due to conditions like colorectal cancer, inflammatory bowel disease, or bladder dysfunction. These bags attach to a stoma, which is an opening created surgically in the abdomen. There are different types of ostomies, including colostomy, ileostomy, and urostomy. Each type corresponds to a different waste elimination function.

Changing an ostomy bag is a routine part of stoma care and helps in maintaining skin health and hygiene. The process typically involves gently removing the old ostomy bag, cleaning the skin around the stoma with warm water and a soft cloth, allowing the area to dry completely, and applying a new ostomy bag. Frequency of changing depends on the type of ostomy, the individual's output, and the specific type of appliance being used, but generally ranges from every few days to once a week. Good technique and proper fitting help prevent complications and support comfort and confidence in daily activities.

### SUMMARY

Apparatus and associated methods relate to an ostomy bag replacement system including an ostomy bag replacement table (OBRT). The OBRT may, for example, include adjustable legs operatively coupled to a bottom surface of the OBRT. The OBRT may, for example, include a top surface. The top surface may, for example, operatively couple two or more waste disposal units. A first at least one waste disposal unit may, for example, include a closed unit. A second at least one waste disposal unit may for example include an aperture. The aperture may, for example, fluidly couple tubing. The top surface may, for example, include a removable and adjustable mirror. The top surface may, for example, include an elevated surface that extends along an outer edge of the top surface. Various embodiments may advantageously provide enhanced convenience, safety, and hygiene for users managing their ostomy care routines.

Various embodiments may achieve one or more advantages. For example, some embodiments may offer improved accessibility and ease of use for individuals with limited mobility or those requiring additional support during the ostomy bag changing process. Some embodiments may, for example, advantageously ensure ergonomic comfort for users of different statures or physical conditions. Some implementations may, for example, provide better visibility for cleaning and monitoring the stoma area, enhancing hygiene and reducing risk of infection. Some embodiments may, for example, prevents spills, minimizing mess and cleanup time. Some implementations may, for example, provide assistance while traveling, promoting independence and flexibility.

The details of various embodiments are set forth in the accompanying drawings and the description below. Other features and advantages will be apparent from the description and drawings, and from the claims.

### BRIEF DESCRIPTION OF THE DRAWINGS

FIG. 1 depicts an exemplary ostomy bag replacement system employed in an illustrative use-case scenario.

FIG. 2 depicts an exemplary schematic of an ostomy bag replacement table (OBRT) with height adjustable legs.

FIG. 3A depicts an exemplary schematic of a second embodiment of an OBRT.

FIG. 3B depicts an exemplary schematic of the second embodiment of the OBRT depicted in FIG. 3A folded and placed in a bag.

Like reference symbols in the various drawings indicate like elements.

### DETAILED DESCRIPTION OF ILLUSTRATIVE EMBODIMENTS

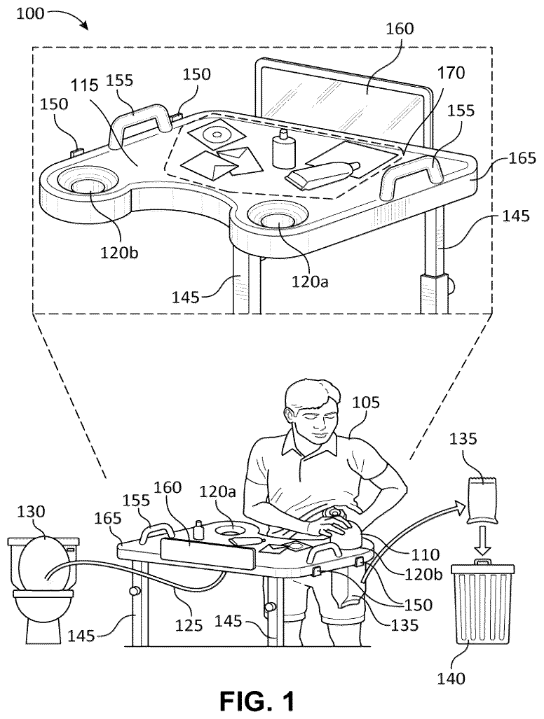

To aid understanding, this document is organized as follows. First, to help introduce discussion of various embodiments, an ostomy bag replacement system is introduced with reference to FIG. 1. Second, that introduction leads into a description with reference to FIGS. 1-3B of some exemplary embodiments of an ostomy bag replacement table (OBRT). Finally, the document discusses further embodiments, exemplary applications and aspects relating to an ostomy bag replacement system.

FIG. 1 depicts an exemplary ostomy bag replacement system employed in an illustrative use-case scenario. The ostomy bag replacement system includes an ostomy bag replacement table (OBRT) 100. A user 105 may, for example, employ an OBRT 100 when changing the user's 105 ostomy bag 110. The OBRT 100 may, for example, include a top surface 115. The top surface 115 may, for example, operatively couple two or more waste disposal units 120a and 120b. At least one waste disposal unit 120a may, for example, include an aperture (not depicted). The aperture may, for example, fluidly couple tubing 125. The fluidly coupled waste disposal unit may, for example, be considered open. Liquid waste from an ostomy bag may, for example, be disposed in at least one disposal unit 120a and through the aperture into the tubing 125. The tubing 125 may, for example, dispose of the liquid waste in a liquid waste disposal site 130. The liquid waste disposal site 130 may, for example, include a toilet, as depicted. The at least one waste disposal unit 120a may, for example, advantageously enable patients that have undergone an ileostomy to dispose of their waste within their ostomy bag.

At least one waste disposal unit 120b may, for example, include a closed unit. For example, the at least one waste disposal unit 120b may, for example, provide a space for a waste disposal bag 135. Waste from an ostomy bag may, for example, be disposed in the waste disposal bag 135 while the waste disposal bag 135 is placed in the at least one waste disposal unit 120b. The waste disposal bag 135 may, for example, be disposed in a solid waste disposal site 140 after waste from an ostomy bag is disposed in the waste disposal bag 135. The solid waste disposal site 140 may, for example, include a trash can, as depicted. The at least one waste disposal unit 120a may, for example, advantageously enable patients that have undergone a colostomy to dispose of their waste within their ostomy bag. The ostomy waste bag disposal unit may, for example, be considered closed.

The two or more waste disposal units 120a and 120b may, for example, be configured to be interchangeable. For example, the two or more waste disposal units 120a and 120b may releasably couple the OBRT 100 such that the user 105 may switch between the at least one waste disposal unit 120a and the at least one waste disposal unit 120b based on the user's specific preferences. The interchangeability of the two or more waste disposal units 120a and 120b may, for example, advantageously enable the customization of the OBRT 100 for different situations, making it more versatile and user-friendly.

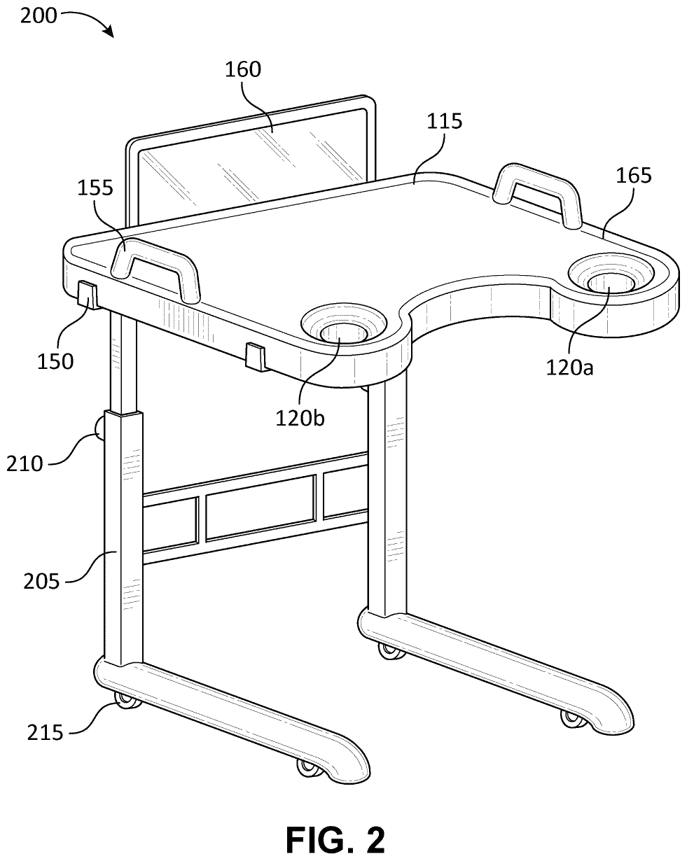

The OBRT 100 may, for example, include two or more legs 145 operatively coupled to a bottom surface of the OBRT 100. As will be described in more detail with reference to FIG. 2, the two or more legs 145 may, for example, include a height adjustable mechanism configured to adjust the height of the two or more legs 145.

The OBRT 100 may, for example, include one or more hooks 150. The one or more hooks 150 may, for example, operatively couple the OBRT 100. The one or more hooks 150 may, for example, advantageously hold a bag that can store the supplies for changing and draining an ostomy bag. The one or more hooks 150 and the bag with all the supplies used for changing and draining an ostomy bag may, for example, advantageously ensure the items are easily accessible during the process of changing an ostomy bag.

The OBRT 100 may, for example, include one or more handles 155. The one or more handles 155 may, for example, operatively couple the top surface 115. The one or more handles 155 may, for example, provide easy lifting of the OBRT 100, making it more convenient to move or adjust. The one or more handles 155 may, for example, enhance the OBRT's 100 portability and usability, especially for individuals who may reposition the OBRT 100 frequently.

The OBRT 100 may, for example, include a mirror 160 releasably coupled to the top surface 115 and extends along one side of an outer edge of the top surface 115. The mirror 160 may, for example, be configured to tilt, rotate, or move to a plurality of positions relative to the top surface 115. For example, the mirror 160 may include a hinge mechanism that enables the mirror to tilt, rotate, or move to a plurality of positions relative to the top surface 115. The mirror 160 may include a swivel mechanism that enables the mirror to tilt, rotate, or move to a plurality of positions relative to the top surface 115, for example. The mirror 160 may, for example, advantageously provide a clear view of the skin around the stoma, making it easier to clean and maintain proper hygiene during the changing process. The adjustability of the mirror 160 allows patients to position it according to their preference. Additionally, the mirror 160 may, for example, advantageously be removed when not in use.

The OBRT 100 may, for example, include an elevated surface 165. The elevated surface 165 may, for example, operatively couple and extend along an outer edge of the top surface 115. The elevated surface 165 may, for example, include bumpers. The elevated surface 165 may, for example, advantageously prevent liquid and solid waste from spilling off the table onto the floor, thereby maintaining cleanliness and reducing the risk of messes during the draining or changing process. The elevated surface 165 may, for example, advantageously ensure that supplies and materials remain securely on the OBRT 100.

The ostomy bag replacement system depicted in FIG. 1 may, for example, include ostomy care supplies 170. By way of example, and not limitation ostomy care supplies 170 may include a new ostomy pouch. By way of example, and not limitation ostomy care supplies 170 may include a skin barrier. By way of example, and not limitation ostomy care supplies 170 may include adhesive remover wipes or spray. By way of example, and not limitation ostomy care supplies 170 may include a stoma measuring guide. By way of example, and not limitation ostomy care supplies 170 may include a soft washcloth or gauze pads. By way of example, and not limitation ostomy care supplies 170 may include disposable gloves. By way of example, and not limitation ostomy care supplies 170 may include a disposable bag.

FIG. 2 depicts an exemplary schematic of an ostomy bag replacement table (OBRT) 200 with height adjustable legs 205. The height adjustable legs 205 may, for example, include a height adjustment mechanism 210. By way of example, and not limitation, the height adjustment mechanism 210 may, for example, include a clamp-and slide mechanism such that the legs have inner sliding segment that can be moved up or down and a clamp locks the leg at the desired height. The height adjustable legs 205 may, for example, operatively couple wheels 215. The height adjustable legs 205 may, for example, advantageously provide flexibility to accommodate different user positions, such as sitting, standing, or wheelchair use, ensuring comfort and ease during the draining and changing processes. This feature is particularly advantageous for patients with limited mobility or balance, enabling them to customize the table height to their specific preferences and reducing physical strain.

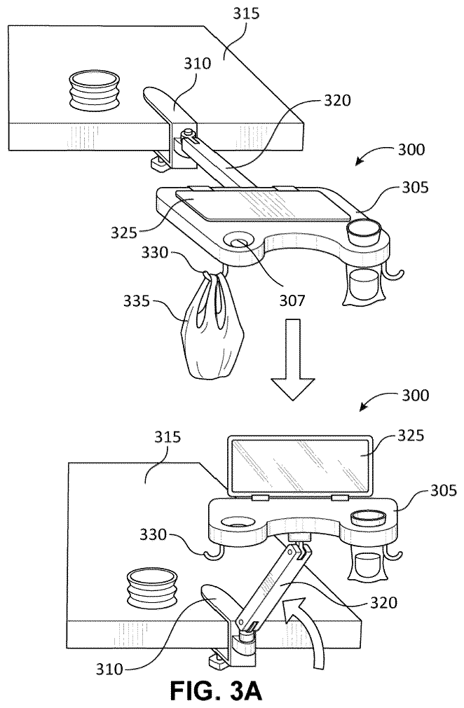

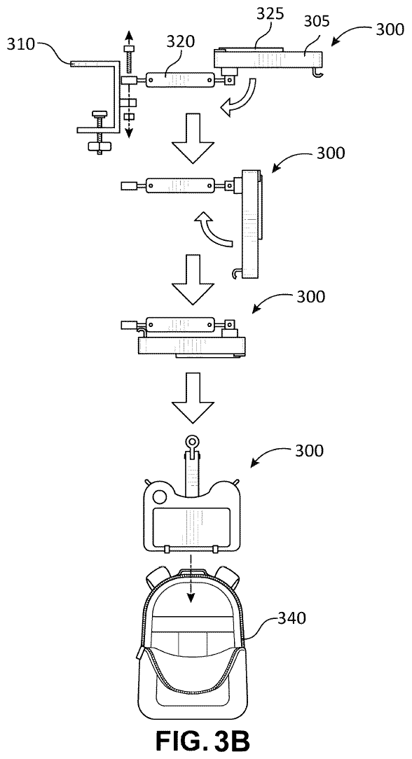

FIG. 3A depicts an exemplary schematic of a second embodiment of an OBRT 300. The OBRT 300 may, for example, include a top surface 305. The top surface 305 may, for example, include at least one waste disposal unit 307. The at least one waste disposal unit 307 may, for example, be configured in substantially the same manner as waste disposal units 120a and 120b. The top surface 305 may, for example, releasably couple a mirror 325 that extends along one side of an outer edge of the top surface. The mirror 325 may, for example, be configured to rotate from a first position, wherein the mirror is oriented substantially vertically, to a second position, wherein the mirror is oriented substantially horizontally. The OBRT 300 may, for example include one or more hooks 330 operatively coupled to the OBRT 300. The one or more hooks 330 may, for example, hold ostomy care supplies (not depicted) during the process of changing an ostomy bag. The ostomy care supplies may, for example, be enclosed by a bag 335. The bag 335 may, for example, advantageously provide an enclosure to keep the ostomy care supplies uncontaminated.

The OBRT 300 may, for example, operatively couple an arm 320 at a distal end of the arm 320. The arm 320 may, for example, releasably couple a clamp 310 at a proximal end of the arm 320. The clamp 310 may, for example, releasably couple an existing object 315. By way of example, and not limitation, the existing object 315 may include an existing table.

FIG. 3B depicts an exemplary schematic of the second embodiment of the OBRT 300 depicted in FIG. 3A folded and placed in a bag 340. As is depicted in FIG. 3B, the arm 320 may, for example, be configured to rotate the OBRT 300 towards the arm 320, thereby reducing the overall profile of the OBRT 300. In this state, the OBRT 300, the clamp 310, and the arm 320 may, for example, be disposed in a bag 340. The bag 340 may, for example include a backpack, as depicted. The rotation of the OBRT 300 into a reduced overall profile may, for example, advantageously, facilitate the portability of an OBRT 300.

Although various embodiments have been described with reference to the figures, other embodiments are possible.

In some embodiments, the ostomy bag replacement table (OBRT) may include adjustable height legs, which may, for example, accommodate the varying preferences of ostomy care. Adjustable legs may, for example, allow for the table to be set at a lower height during the draining process, which may, for example, require the user to be seated or in a bent-over position.

In a changing the ostomy bag mode, the table may, for example, be adjusted to a higher setting, facilitating easier access and reducing strain on the user. The ability to adjust height may, for example, ensures that the table may be customized to the comfort and physical capabilities of individual users, enhancing the overall experience of ostomy maintenance.

In some embodiments, the OBRT may include interchangeable waste disposal unit holders designed to accommodate different types of ostomy bags, including colostomy and ileostomy bags. This versatility is provided by offering two distinct waste disposal options: a closed waste disposal unit for direct waste disposal into a trash receptacle and a draining tube waste disposal unit that can be connected to a toilet, facilitating seamless waste management. The waste disposal sites may, for example, be used in connection with the table's adaptability to the user's preferences and requirements, making the process of draining and/or changing ostomy bags more efficient and hygienic.

In some embodiments, the OBRT may include a removable and adjustable mirror, which is a thoughtful addition to aid users in accurately and safely managing their stoma care. The mirror allows for a full, clear view of the stoma area, enabling users to perform cleaning and maintenance with greater precision and confidence. This feature may, for example, particularly benefit a user to ensuring proper skin care around the stoma, thereby reducing the risk of skin irritation and infections, which may, for example, be common concerns among ostomy patients.

In some embodiments, the OBRT may come in various design options to cater to the diverse lifestyles and preferences of its users. These options range from a portable travel version with a collapsible waste disposal unit for ease of transport to a stationary wall-mount model designed for fixed use in a bathroom setting. Additionally, an extender for wheelchair users enhances accessibility, and a single stand/pedestal version offers focused support for draining. Each design variation addresses specific user scenarios, ensuring that the table remains a convenient and accessible tool for ostomy patients, regardless of their mobility level or living situation.

In some embodiments, the OBRT may be constructed from lightweight, non-porous materials such as medical-grade silicone and corian. These materials are selected for their durability, ease of cleaning, and hygienic properties, aligning with the healthcare requirements for medical devices. The non-porous nature of these materials ensures that the table remains sanitary and resistant to bacteria buildup. The non-porous nature may, for example, be a consideration for medical equipment used in ostomy care. This focus on material selection highlights the table's design philosophy of prioritizing user safety and hygiene.

In some embodiments, the OBRT may, for example, feature several design innovations aimed at enhancing user convenience and safety. These include a raised lip around the table's edge to prevent spillage, hooks for hanging a supply bag, and a flat, non-slip surface for securely organizing ostomy care supplies. These features may, for example, collectively contribute to a safer, more organized, and efficient process for changing or draining ostomy bags, demonstrating a comprehensive approach to addressing the practical challenges faced by ostomy patients.

In some embodiments, the design of the OBRT may, for example, incorporate bumpers along its edges. The bumpers may, for example, be used for spillage containment. The bumpers may, for example, provide a cushioned barrier that allows users to safely and comfortably press against the table during the ostomy care process. This feature may, for example, be beneficial for users who prefer to lean in or stabilize themselves against the table while changing or draining their ostomy bags. The bumpers may, for example, be made from soft, durable materials that can withstand pressure without causing discomfort to the user. The bumpers may, for example, be used to support the user's safety and comfort, ensuring that individuals can engage in their care routines with added support and without the risk of injury from hard surfaces. By allowing users to press against the table securely, the bumpers may, for example, enhance the functionality of the table, making it a more versatile and user-friendly tool in the management of ostomy care.

In travel mode embodiments, the OBRT may, for example, be designed with portability and compactness in mind, addressing the preferences of users who require ostomy care while on the move. These embodiments may, for example, feature a collapsible design, with parts like the waste disposal unit folding down and/or accordion-style for easy packing. Lightweight materials may, for example, be used to ensure the table can be transported without significant burden. Additionally, travel mode tables may, for example, include detachable components and/or fold-in-half functionalities, allowing them to fit into luggage or carry-on bags conveniently. This focus on mobility enables users to maintain their ostomy care routines without compromise, providing peace of mind during travel and ensuring that users can manage their healthcare with dignity and privacy, regardless of their location.

Stationary embodiments of the OBRT may, for example, be designed for fixed use, often in a bathroom or dedicated care area within the user's home. These versions may include features such as wall-mounting capabilities, allowing the table to be securely attached to a bathroom wall and folded down. The stationary embodiments may, for example, optimize space utilization, making the ostomy table a helpful yet unobtrusive part of the home healthcare environment. Stationary tables can also be equipped with more permanent fixtures, such as built-in plumbing connections for the draining waste disposal unit, enhancing the efficiency and hygiene of the waste disposal process. The focus on stationary use ensures that users have a reliable, sturdy platform for ostomy care, providing stability and confidence during the changing or draining process.

Table embodiments of the OBRT may, for example, be used for a blend of stability, accessibility, and comprehensive care functionality. Table embodiments may, for example, be designed with a flat, stable surface area that accommodates the supplies used for ostomy care, including spare bags, cleaning materials, and personal hygiene items. The inclusion of features such as adjustable height, interchangeable waste disposal units, and organizational hooks and compartments allows users to customize the table to their specific preferences, fostering a sense of autonomy and efficiency in their care routine. Furthermore, the emphasis on durable, easy-to-clean materials ensures that the table remains a hygienic, safe, and long-lasting tool for ostomy management. By addressing the varied and complex preferences of ostomy care, table embodiments offer a holistic solution that supports users in maintaining their health and wellbeing with dignity and ease.

In an illustrative use-case scenario, as depicted in FIG. 1, the user may approach the OBRT to change their ostomy bag, leveraging the table's adjustable height to position it comfortably at waist level. The user may press gently against the cushioned bumpers for stability as they lean in, using the table's flat, non-slip surface to lay out their supplies: a new ostomy bag, skin barrier wipes, and a disposal bag for the used appliance. The user may, for example, appreciate the convenience of having ostomy care supplies within reach, thanks to the organizational hooks and compartments. After removing the old bag, the user may, for example, clean the stoma area using the supplies laid out, occasionally glancing in the adjustable mirror to ensure thorough cleaning around the stoma. The process may, for example, be completed with the attachment of a fresh ostomy bag, made easier and more comfortable by the supportive and hygienic environment provided by the table.

In an illustrative use-case scenario, as depicted in FIG. 1, in a separate instance, a user may, for example, employ the OBRT's drainage system to empty their ostomy bag directly into the toilet. The user may, for example, adjust the table to a lower height for ease of access. The user may, for example, position the draining tube waste disposal unit, which is attached to the toilet bowl, underneath their ostomy bag's outlet. The user may, for example, lean slightly against the table, finding support against the soft bumpers, ensuring a stable stance without having to overly bend or strain. The ostomy table setup may, for example, allow a user less hassle as the contents of the bag is directed into the toilet through the tubing, significantly reducing the risk of spillage and contamination. The ease of the process may, for example, be enhanced by the table's design, which may, for example, accommodate the user's preferences for stability, hygiene, and convenience during the draining procedure.

Although an exemplary system has been described with reference to FIGS. 1-3B, other implementations may be deployed in other industrial, scientific, medical, commercial, and/or residential applications.

The ostomy bag replacement table (OBRT) may, for example, be used in industrial settings, such as healthcare facilities, nursing homes, and rehabilitation centers, where efficient and hygienic waste management is an important parameter. The OBRT embodiments including a waste disposal unit drainage system and built-in cleaning supplies, may, for example, enhance the safety and comfort of patients while minimizing the physical strain on healthcare workers. The OBRT may, for example, improve operational efficiency and hygiene standards in these environments.

In scientific research, the OBRT may, for example, be used to study the ergonomics and efficiency of ostomy care practices. The OBRT features, such as the detachable mirror and soft bumpers, may, for example, provide a basis for examining how design innovations can improve self-care for individuals with ostomies. Research could explore the effectiveness of OBRT embodiments in reducing contamination risk, improving user comfort, and facilitating independence, potentially contributing to the development of guidelines for ostomy care equipment design and usage.

Medically, the OBRT may, for example, enhance patient care through improved ostomy management. By facilitating a safer, more comfortable, and hygienic process for changing and draining ostomy bags, the OBRT may, for example, reduce the risk of skin irritations and infections associated with ostomy care. For patients in hospitals or receiving home healthcare, the OBRT may, for example, improve the quality of life, offering them a degree of independence and dignity in managing their condition.

Commercially, the OBRT may, for example, offer opportunities for businesses specializing in medical supplies and equipment. The OBRT's portability, ease of use, and adaptability may, for example, make the OBRT an attractive product for retailers. The OBRT may, for example, be used to provide businesses the chance to expand their offerings with a product that enhances customer satisfaction and loyalty among users requiring ostomy care solutions.

For residential settings, the OBRT may, for example, be used to provide individuals managing an ostomy with a tool for daily care. The OBRT's adaptability for home use, including versions that are foldable or fixed, may, for example, be used to users to integrate the table seamlessly into their living spaces. The convenience of having a dedicated, ergonomically designed space for ostomy care may, for example, empower users. The OT may, for example, allow users to make self-care routines less daunting and more manageable, thus improving their overall wellbeing and autonomy in personal healthcare management.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. An ostomy bag replacement table comprising:
a plurality of waste disposal units extending along a top surface of an ostomy table configured such that the plurality of waste disposal units releasably couple to a corresponding plurality of apertures extending the through the top surface of the ostomy table;
wherein the plurality of waste disposal units comprise at least one closed waste disposal unit configured to releasably couple to at least one corresponding opened ostomy waste bag enclosing a closed waste disposal aperture configured to receive ostomy waste through the closed waste disposal aperture into the corresponding opened ostomy waste bag; and,
wherein the plurality of waste disposal units comprise at least one open elevated waste disposal unit configured to releasably couple to a receiving end of a corresponding tubing fluidly coupled to receive fluid ostomy waste through the receiving end so the liquid ostomy fluid waste travels through the tubing to an opposing tubing end to be disposed of into a lower elevation liquid waste disposal site;
a plurality of height adjustable legs operatively coupled to a bottom surface of the table configured such that a user may adjust the ostomy table to their height;
two or more opposing handles operatively coupled to the top surface configured such that the user may grip the handles when using the ostomy table;
one or more hooks operatively coupled to the table configured to stow waste disposal bags; and,
an adjustable mirror releasably coupled to the top surface that extends along an opposing end of an outer edge of the plurality of waste disposal units.

2. An ostomy bag replacement table comprising:
a plurality of waste disposal units extending along a top surface of an ostomy table configured such that the plurality of waste disposal units releasably couple to a corresponding plurality of apertures extending the through the top surface of the ostomy table;
wherein the plurality of waste disposal units comprise at least one closed waste disposal unit configured to releasably couple to at least one corresponding opened ostomy waste bag enclosing a closed waste disposal aperture configured to receive ostomy waste through the closed waste disposal aperture into the corresponding opened ostomy waste bag; and,
wherein the plurality of waste disposal units comprise at least one open elevated waste disposal unit configured to releasably couple to a receiving end of a corresponding tubing fluidly coupled to receive fluid ostomy waste through the receiving end so the liquid ostomy fluid waste travels through the tubing to an opposing tubing end to be disposed of into a lower elevation liquid waste disposal site.

3. The ostomy bag replacement table of claim 2, further comprising a plurality of height adjustable legs operatively coupled to a bottom surface of the table configured such that a user may adjust the ostomy table to their height.

4. The ostomy bag replacement table of claim 3, further comprising a plurality of rollers couple to the height adjustable legs.

5. The ostomy bag replacement table of claim 2, further comprising two or more opposing handles operatively coupled to the top surface configured such that the user may grip the handles when using the ostomy table.

6. The ostomy bag replacement table of claim 2, further comprising one or more hooks operatively coupled to the table configured to stow waste disposal bags.

7. The ostomy bag replacement table of claim 7, wherein the hooks are configured to stow ostomy care supplies.

8. The ostomy bag replacement table of claim 2, further comprising an adjustable mirror releasably coupled to the top surface that extends along an opposing end of an outer edge of the plurality of waste disposal units.

9. The ostomy bag replacement table of claim 2, further comprising an adjustable mirror affixed to the top surface that extends along an opposing end of an outer edge of the plurality of waste disposal units.

10. The ostomy bag replacement table of claim 2, further comprising a hinged mirror affixed to the top surface that extends along an opposing end of an outer edge of the plurality of waste disposal units configured to operate in an operation mode and a stowed mode when folded.

11. The ostomy bag replacement table of claim 2, wherein the disposable bags when filled with ostomy waste may be decoupled to be disposed into a solid ostomy waste disposal site.

12. An ostomy bag replacement table:
a plurality of waste disposal units extending along a top surface of an ostomy table configured such that the plurality of waste disposal units releasably couple to a corresponding plurality of apertures extending the through the top surface of the ostomy table;
an adjustable arm coupled to a rear bottom portion of the ostomy table at a first end, the opposing end to coupled to a preexisting object, wherein in an adjustment mode a user may adjust the height of the adjustable arm to adjust the height of the ostomy table to the height of the user;
wherein the plurality of waste disposal units comprise at least one closed waste disposal unit configured to releasably couple to at least one corresponding opened ostomy waste bag enclosing a closed waste disposal aperture configured to receive ostomy waste through the closed waste disposal aperture into the corresponding opened ostomy waste bag; and,
wherein the plurality of waste disposal units comprise at least one open elevated waste disposal unit configured to releasably couple to a receiving end of a corresponding tubing fluidly coupled to receive fluid ostomy waste through the receiving end so the liquid ostomy fluid waste travels through the tubing to an opposing tubing end to be disposed of into a lower elevation liquid waste disposal site.

13. The ostomy bag replacement table of claim 11, further comprising an ostomy table storage mode, wherein the ostomy adjustable arm may be adjusted to be secured to the bottom of the ostomy table.

14. The ostomy bag replacement table of claim 11, wherein in the storage mode the ostomy table is stowed into a backpack.

15. The ostomy bag replacement table of claim 11, further comprising one or more hooks operatively coupled to the table configured to stow waste disposal bags.

16. The ostomy bag replacement table of claim 15, wherein the hooks are configured to stow ostomy care supplies.

17. The ostomy bag replacement table of claim 11, further comprising an adjustable mirror releasably coupled to the top surface that extends along an opposing end of an outer edge of the plurality of waste disposal units.

18. The ostomy bag replacement table of claim 11, further comprising a hinged mirror affixed to the top surface that extends along an opposing end of an outer edge of the plurality of waste disposal units configured to operate in an operation mode and a stowed mode when folded.

19. The ostomy bag replacement table of claim 11, wherein the disposable bags when filled with ostomy waste may be decoupled to be disposed into a solid ostomy waste disposal site.

20. The ostomy bag replacement table of claim 11, wherein the plurality of waste disposal units are interchangeable configured such that the plurality of waste disposal units may be removed.
